(19) United States
(12) Patent Application Publication
(10) Pub. No.: US 2002/0001595 A1
(43) Pub. Date: Jan. 3, 2002

(54) Process for the preparation of antiviral agents

(76) Inventors: Hans-Gunther Sonntag, Bammental (DE); Oliver Nolte, Leimen (DE); Hannelore Weiss, Neckargemund (DE); Hans-Erich Weiss, Neckargemund (DE)

Correspondence Address: Michele M. Simkin, FOLEY & LARDNER, Suite 500, 3000 K Street, N.W., Washington, DC, 20007-5109, US

(21) Appl. No.: 09/839,592

(22) Filed: Apr. 23, 2001

(30) Foreign Application Priority Data
Oct. 9, 1999 (EP) PCT/EP99/07588
Oct. 28, 1998 (DE) 198 49 641.9
May 3, 2000 (DE) 100 21433.9

(51) Int. Cl. A61K 39/21; A61K 39/29; A61K 39/245; A61K 39/255

(52) U.S. Cl. 424/208.1; 424/225.1; 424/229.1

(57) ABSTRACT

The present invention is directed to a process for preparing an antiviral agent in which antigen-containing blood and/or tissue is heated to a temperatures above about 50° C. in the presence of at least one protein cross-linking agent, such as formaldehyde, p-formaldehyde, formalin, phenol, and/or phenol derivatives.

### FIELD OF THE INVENTION

The present invention is directed to a novel process for preparing an antiviral agent.

### BACKGROUND OF THE INVENTION

It is known that viruses cannot be combated, or only insufficiently challenged, in the human or the animal body. Thus, up until now it has not been possible to effectively treat or cure HIV-positive patients. This can be attributed to the fact that viruses, such as HIV, are able to mutate such that the immune system mechanisms which combat the original virus no longer attack the mutated virus due to changes in the genetic and protein structure of the mutated virus.

U.S. Pat. No. 5,698,432, for “Vaccines and Methods for Their Production,” refers to preparing antiviral vaccines by inactivating cultured viruses with propiolactone, followed by separating the inactivated viruses from the culture liquid. The viruses are deaggregated and the virus cover is distended, preferably with solvents and detergents, to subsequently inactivate the viral RNA with ethyleneimine and RNAse/DNAse. Viruses prepared using this method are stabilized with formaldehyde and diluted with adjuvants to provide vaccine standards.

One drawback to prior art methods of preparing antiviral preparations is that the resulting preparations cannot act specifically against mutated viral strains.

There is a need in the art for new methods of making antiviral compositions. The present invention satisfies this need.

### SUMMARY OF THE INVENTION

The present invention is directed to the surprising discovery that safe and effective antiviral preparations which specifically attack variant forms of viruses can be made.

The compositions of the invention are made by heating virus- and antigen-containing blood and/or tissue in the presence of protein cross-linking agents, such as formalin, formaldehyde, p-formaldehyde, phenol, and/or phenol derivatives, to temperatures above about 50° C. The resulting formulation is liquified, and if needed sterilized, for use as a therapeutic.

Another aspect of the invention encompasses treatment of a patient in need with an antiviral composition of the invention. Such a method comprises administering to a patient in need an effective amount of an antiviral preparation of the invention in one or more doses over a period of time.

It is to be understood that both the foregoing general description and the following detailed description are exemplary and explanatory and are intended to provide further explanation of the invention as claimed. Other objects, advantages, and novel features will be readily apparent to those skilled in the art from the following detailed description of the invention.

### DETAILED DESCRIPTION OF THE INVENTION

The present invention is directed to a novel method of preparing an antiviral agent which is an autovaccine. The term autovaccine generally refers to a therapeutic formulation useful against infections, such as for example chronic bacterial infections, in which causative infectious agents are taken from the site of infection, obtained as a pure culture, and subsequently modified physically and/or chemically to form a composition comprising an inactivated form of the infectious agent.

The present invention is directed to methods of making autovaccines for viral diseases in which the viral causative agent is present in whole blood, in parts of blood as lymphocytes or serum, in tissue, or in a combination of blood and tissue. The method comprises heating the viral-containing blood and/or tissue to denature the infectious viral agent (i.e., the antigen) in the presence of a cross-linking agent to obtain an autovaccine for the viral infection.

By the treatment of the patient's own blood and/or tissue at elevated temperatures in the presence of cross-linking reagents, all proteins contained in the blood are individually denatured and cross-linked. This denaturing plus cross-linking results in a virus (i.e., the causative agent) which is non-native (i.e., not infectious) but yet accessible to the immune system. Surprisingly, administration of the resultant autovaccine to the patient, in one or multiple doses, results in suppression of the viral infection by a specific immune response generated against the denatured viral particles of the autovaccine.

Useful protein cross-linking agents include, for example, formalin, formaldehyde, paraformaldehyde, phenol, and/or phenol derivatives. A useful amount of a protein cross-linking agent such as formalin is, for example, at least about 0.1 to about 1.0 volume percent in the form of a saturated formalin solution.

The denaturing temperature is generally above about 50° C., preferably above 55° C., and most preferably between about 80 and about 85° C. The elevated temperature is preferably maintained for about 2 hours.

The autovaccine can be administered via any conventional method, such as subcutaneously, perorally, or buccally. For preparation of a vaccine to be administered subcutaneously, the material treated according to the invention is filtered over filters with pore sizes of about 400 μm. The liquefied agent can also be administered to the patient via the mucous membranes of the mouth (i.e., via gargling).

Using the method of the invention, infectious viral agents which possibly remain unknown in the native state, or induce an inadequate form of the immune response (chronic inflammatory course or the like), can be treated or prevented. Examples of such viral diseases include, for example, various tumors or carcinomas having a viral cause, such as certain sarcomas, melanomas, sarcoids, cervix carcinomas, etc., or regionally limited viral tissue diseases, such as the Crohn's disease, which is localized in the small intestine.

Autovacines against lymphotropic viruses found in whole blood can also be made according to the method of the invention. Such viruses include HIV, the various HIV viral forms found in infected cells, HIV viral forms existing after lysis of infected cells, and HIV viral forms existing in interactions with certain cell components or cell receptors.

These considerations are supported by extensive investigations with animal and human patients. The autovaccine is particularly useful for chronically persisting or recidivising (relapsing) infections. After administration of the antigen in denatured and cross-linked form, the autovaccine produced, in most cases in less than four weeks, healing or a dramatic decrease in the severity of the infection. This can only be explained by the described changed form of presentation of the antigen by heat and cross-linking.

The experimental data indicate that a change in the immune response takes place after administration of the autovaccine. This change, simply illustrated, consists in an exchange from an inflammatory (Th-1) to a helper cell-mediated (Th-2) response. On the basis of the Th-2 response, the presence of the viral or infectious agent, which had previously led to a chronic inflammatory reaction, can be eliminated or dramatically decreased. In the case of HIV, it is postulated that administration of the autovaccine according to the invention results in a protecting cytotoxic T-cell reaction and simultaneously changes the antibody quality.

Following is a summary of a method of preparing an antiviral preparation according to the invention from blood and from tissue.

A. Antiviral Preparation from a Blood Sample

The blood removed is kept liquid during the removal and thereafter by mechanical action or by chemical coagulation inhibitors, such as, e.g., EDTA, heparin, or hirudin, to ensure a good distribution of the cross-linking agent.

The mechanical maintenance of the flowability of the blood following removal and thereafter, i.e. the destruction of fibrin, can be carried out in a conventional method, such as by shaking in the presence of glass pearls.

The viral protein in the blood sample is denatured by heating the sample to above about 50° C., preferably above about 55° C., and most preferably between about 80 and about 85° C., for about 2 hours. The denaturing is performed in the presence of at least one cross-linking agent, such as formalin, formaldehyde, paraformaldehyde, phenol, and/or phenol derivatives.

The denaturing treatment results in a solidified blood sample. Prior to administration, the solidified blood is liquefied. An exemplary liquification process comprises adding a pyrogen-free physiological common salt solution with stirring to the autovaccine. The resultant autovaccine viral particles induce an anti-viral agent-specific immune response upon administration.

If the viruses to be combated are bound to lymphocytes, i.e., the viruses are present only in small amounts in the accompanying erythrocytes and in the serum, a virus enrichment can be achieved. In this embodiment of the invention, the erythrocytes are lysed in a known manner, and the serum and lysed erythrocytes are then separated from the lymphocytes by centrifugation. By resuspending the lymphocyte fraction in physiological common salt solution or in phosphate buffered saline (PBS), a suitable virus concentration can be produced which then, as with whole blood, is treated with at least one cross-linking agent in the presence of an elevated temperature to prepare the autovaccines according to the invention.

For viruses which occur in a high concentration in the blood serum (e.g., hepatitis B and C viruses, the origin of which is the liver, as well as other comparable viruses which occur in the blood but do not have their origin in blood cells), lysis of the erythrocytes is not required.

A further modification of the process of the invention is represented by the separation of the lymphocyte fraction from the viruses present in the blood by centrifugation of the lymphocytes after lysis of the erythrocytes. Such centrifugation can be for about 10 min.

Subsequently, the viruses and lymphocytes are taken up in culture. After virus culturing has taken place, the prepared viruses are used for infection of cultured lymphocytes. After a period of time, such as e.g., several days, the infected cultured lymphocytes are treated according to the preparation procedure of the autovaccines of the invention.

The viruses can be purified via routine preparation techniques, and the culture of the lymphocytes also presupposes established methods for the cell culture. The culture media should have serum-free supplements or contain as a protein source inactivated serum obtained from the patient.

This method is applicable for, e.g., hepatitis B and C viruses. The object of this treatment is to match to the greatest extent possible the autovaccines to the in vivo conditions. This means that a virus as an infectious and causative agent of a chronic/persisting/recidivising infection is denatured and cross-linked, and that the immune cells which come into contact with such a virus, as well as the surface receptors for the antigen presentation expressed on these immune cells, are denatured and cross-linked. Via the separation of the lymphocytes, there is achieved a “better” appearance of the autovaccines.

Ultrasonic treatment of the lymphocytes obtained as described above represents an additional modification of the process of the invention. Using conventional ultrasonic techniques, this method results in destruction of the cells, and thus fractionation of the virus proteins, as well as of the surface receptors associated with the virus proteins. In the subsequent denaturing and cross-linking, these fractions also undergo cross-linking.

B. Antiviral Preparation from Tissue Sample

For viral diseases in which the viruses occur only insufficiently in the blood, samples of diseased virus-containing tissue can be used for preparation of an autovaccine according to the invention.

In this embodiment of the invention, a sample of a patient's virally-infected tissue is removed, mixed with an aqueous dilution agent, homogenized, and treated at an elevated temperature in the presence of one or more cross-linking agents to prepare an autovaccine of the invention.

The tissue removed is kept liquid during the reaction and thereafter by mechanical action or by chemical coagulation inhibitors, such as e.g., EDTA, heparin, or hirudin, to ensure a good distribution of the cross-linking agent.

In an exemplary process, a patient's virus-containing tissue is removed in an amount of a few cubic centimeters, mixed with about 1 to about 5 times of an amount of physiological common salt solution or another physiologically compatible aqueous dilution agent, homogenized by mechanical action, and treated at elevated temperatures in the presence of at least one cross-linking reagent. Homogenization can be accomplished by, for example, ultrasonic treatment or by using a rapidly-running grinder. Useful cross-linking agents include, for example, formalin, formaldehyde, paraformaldehyde, phenol, and/or phenol derivatives.

The proteins in the tissue homogenate are thereby together cross-linked and denatured, i.e., the virus or antigen in the tissue is acted upon in a specific way which surprisingly forms an antiviral agent which, upon administration, leads to virus suppression or dramatic reduction.

The following examples are given to illustrate the present invention. It should be understood, however, that the invention is not to be limited to the specific conditions or details described in these examples. Throughout the specification, any and all references to publicly available documents are specifically incorporated into this patent application by reference.

### EXAMPLE 1

The purpose of this example was to describe preparation of an autovaccine of the invention.

100 ml of blood were introduced into a sterile 500 ml flask having about 50 sterile glass pearls (diameter 3-5 mm). The flask was shaken during addition of the blood for defibrination, and after the blood sample was completely added to the flask it was further shaken for 10 minutes. Following this procedure, the blood sample remained liquid.

Next, 0.5 ml of a saturated, chemically pure formalin solution was added to the flask, followed by shaking to mix the blood/formalin composition. Thereafter, the flask was placed in a water bath and the water temperature was slowly brought to 80° C. to 85° C. This elevated temperature was maintained for 2 hours, after which the blood sample solidified.

Subsequently, 200 ml of sterile, pyrogen-free physiological NaCl solution was added to the solid (chocolated) blood, and the flask was shaken until the glass pearls were again free and the mass optically homogeneous and liquid. Thereafter, the flask contents were applied to a sterile sieve (edge lengths of the meshes about 400 μm) and passed through with a sterile spoon.

In addition to the blood sample, the flask contained 66% phys. NaCl solution and 0.5 ml of the saturated formalin solution. The autovaccine preparation was subsequently incubated for 24 hours at 37° C. Prior to use, the sterility of the autovaccine is verified.

### EXAMPLE 2

The purpose of this example was to determine the efficacy of an autovaccine against HIV prepared as described in Example 1.

For the autovaccine treatment, a volunteer patient was selected who, according to information of his treating specialist, had “Stage C₃ HIV infection with thrombocytopenia” (“Stage C₃” is a standard set forth by the Centers for Disease Control (CDC)). In addition, the patient had a chronically persisting hepatitis C infection, and additional accompanying diseases, including, inter alia, an atypical myco-bacteriosis infection. For ethical and medical reasons, it was not acceptable to withdraw simultaneous treatment with antiviral agents during the autovaccine therapy.

The patient had the following preliminary HI virus loading values with conventional antiviral medicament treatment:

| | |
|---|---|
| physician's letter | 3700/μl |
| determination 3 months later | 2500/μl |
| (before beginning of the autovaccine | |
| therapy) | |

As described above, an autovaccine was prepared with whole blood of the patient. The administration took place subcutaneously and perorally following a specified scheme: 3 ml were administered subcutaneously at days 1, 5, 10, and 15; and 10 ml was administered perorally for 10 days starting with the first day.

Heparinised whole blood was taken from the patient before the beginning of treatment, and at 7 days, three weeks, and eight weeks after the first administration of the autovaccine. The lymphocytes therefrom were purified according to standard procedures over ficoll, and the serum was removed and frozen. The relative proportions of the CD4-, CD8-, CD21-, and CD3- (not at the 7th day) positive cells were determined in a flow-through cytometer using the lymphocytes and a specific monoclonal antibody (obtained from Cymbus Lab., US). After conclusion of the experiment, the neopterin value was determined from the serum.

For the lymphocyte preparation, the first to the fourth measurement showed a clear increase of cell yields per ml of whole blood (although this can naturally vary), whereby the proportion of contaminated cells, such as granulocytes and thrombocytes, decreased distinctly. Moreover, during the course of treatment, an increase in the number of CD4-, CD8-, and CD3-positive cells was shown. Specifically, the CD8-positive cells clearly increased above the normal value. In addition, three weeks after beginning the autovaccine treatment, CD21-positive cells increased, following which the cell level fell but still remained in the normal range. Finally, the CD4/CD8 index was measured at 0.5 (norm: 1.0-2.3), but the proportion of the CD8-positive cells was far above the normal value (49.9% measured by an independent laboratory, whereas 17-35% is the normal value). This high percentage of CD8-positive cells is generally a characteristic of a strengthened cytotoxic defense against intracellular pathogens, preponderantly viruses.

The neopterin value (parameter for the course control of viral, as well as intracellular infections) in the serum was increased before administration of the autovaccine, varied during the course of the investigations, and after termination of the autovaccine administration remained higher than at the beginning of the experiment. (However, the neopterin level is influenced by mycobacterial infections or generally by inflammatory processes of the Th1 type, in which interferon γ is liberated.)

In addition, the patient showed positive physiological reactions. About 30 h after the first administration of the vaccine, the patient reacted with subfebrile temperatures (but no inflammatory indications at the point of injection) and slight diarrhea, which permitted the conclusion of an immune reaction. During the following weeks, the patient showed a continuing weight increase as well as a distinct general improvement.

The viral HI load after conclusion of the therapy was <50/μl, which corresponds to the normal viral range. This shows a dramatic improvement in the patient's viral load present with conventional antiviral treatment prior to autovaccine treatment of 2500/μl. The viral load value of <50/μl was determined in four investigations, independent of each other.

The patient remained positive for HIV provirus DNA (gene region gag). However, this finding merely indicates that proviral DNA is present. No statement can be made about the actual virus loading or the status of a florid infection, as viral RNA is measured to determine virus load. After cell death, liberated DNA can, under certain circumstances, be detectable for very long. Thus, the presence of viral DNA may not correspond to a continued HIV infection.

In sum, the autovaccine demonstrated remarkable effectiveness in treating an HIV positive patient.

### EXAMPLE 3

The purpose of this example was to prepare and test the effectiveness of an autovaccine according to the invention for Crohn's disease caused, at least in part, by human herpes virus Type 6.

Preparation of the Autovaccine

An autovaccine according to the invention was prepared for treatment of the chronic inflammatory disease of the small intestine Crohn's disease. The autovaccine preparation comprised the human herpes virus Type 6 (HHV6), which according to current knowledge is at least partly responsible for Crohn's disease. See e.g., A. J. Wakefield et al., J. Med. Virol., 38(3):183-190 (1992).

A 4 cubic centimeter tissue sample was endoscopically-obtained from the inflamed small intestine mucous membrane of the patient. To improve passability, the small intestine mucous membrane was mixed with a 3 fold amount of a physiological common salt solution (alternatively, a commercially available cell culture medium, e.g., RPMI 1640 without serum addition or other media suitable for the cell culture, can be used) and homogenized by mechanical action (ultrasonic treatment).

Thereafter, the tissue sample was mixed with 0.3 vol. % of formalin, heated at 80° C. for 2 hours, and incubated for 24 hours at 37° C. The preparation was then passed through a sterile sieve (pore width of 400 μm). The preparation was tested for sterility (negative), and was then ready for use.

Administration of the Autovaccine

A 38 year old patient, who has suffered from chronic recidivising Crohn's disease for 19 years, was autovaccinated. During a severe recidivate phase, an abscess was sonographically detected in the left lower abdomen. Prior to autovaccination, HHV6 was positively detected by PCR diagnosis of mucous membrane tissue of the small intestine.

At initiation of treatment, about 2 ml of the autovaccine was administered subcutaneously. Following the initial administration, 2 ml of the autovaccine was administered at 5, 10, and 15 days. The amount of material which can be obtained endoscopically is sufficient to carry out a four administration injection cycle. The autovaccine is patient specific.

For about 4 weeks after beginning the autovaccination, the treated patient showed a significant improvement of the general state of health with weight increase (6 kg), a macroscopic clear improvement of the condition, and a sonographic almost complete remission of the initially fist-sized inflammation characterizing Crohn's disease, with only a few remaining inflammatory mucous membrane changes. PCR detection of HHV6 was only weakly positive.

According to information from the treating physician, a spontaneous remission can be excluded. This conclusion is supported by the local immunological reaction at the injection site on the thigh (probably so-called DTH reaction) and by the occurrence of pain after all four administrations in the region of the abscess in the left lower abdomen, which indicates a rapid immunological reaction (e.g., activation of specific cytotoxic cells).

In sum, the autovaccine demonstrated remarkable effectiveness in treating a patient suffering from Crohn's disease.

### EXAMPLE 4

The purpose of this example was to prepare and test the effectiveness of an autovaccine according to the invention for equine sarcoids (most benign cutaneous tumors) caused by papilloma viruses.

Preparation of the Autovaccine

A patient-specific autovaccine was prepared from virus-containing tumor material obtained by surgical intervention. The tissue was first mechanically sliced using a scalpel (or razor blade, etc.), followed by subjecting the tissue to high shear mixing using a Ultra-Turrax® apparatus at 20,000 r.p.m. for a few seconds to obtain a homogeneous suspension.

The density of the autovaccine preparation was then adjusted to a McFarland density of about 5-6 using a sterile, pyrogen-free common salt solution. Further treatment with the cross-linking agent formalin and heating took place as in Example 3.

Administration of the Autovaccine

More than 100 animals were treated with an autovaccine preparation according to the invention for the prophylaxis of post-operative equine tumor recidivities. The treatment protocol consisted of 5 ml of autovaccine administered subcutaneously four times at five-day intervals (i.e., at 0, 5, 10, and 15 days).

More precise data was obtained for 31 of the animals from eleven of the treating veterinary surgeons. The average age of the sample of 31 treated animals at the time of autovaccination was 7.83+/−0.76 years.

For 21 of the 31 treated animals, autovaccination took place as a first treatment after operation. For prevention of possible recidivity, the remaining 10 of the 31 animals were treated after the appearance of post-operative recidivity with an autovaccine prepared from the operatively excised reciditive material. For 27 of the treated 31 animals (87.1%), no further recidivity was observed after autovaccination during the observation period of 15.133 months +/−1.5 months. For the four animals which, in spite of autovaccination, showed renewed recidivity, there was no relationship between the appearance of recidivity and the period of observation. For 2 of the 4 animals showing recidivity, the recidivising sarcoids were distinctly smaller than the primary recidivised sarcoids.

In sum, the autovaccine demonstrated remarkable effectiveness in treating animals suffering from equine sarcoids.

It will be apparent to those skilled in the art that various modifications and variations can be made in the methods and compositions of the present invention without departing from the spirit or scope of the invention. Thus, it is intended that the present invention cover the modifications and variations of this invention provided they come within the scope of the appended claims and their equivalents.

We claim:

1. A process for preparing an antiviral agent comprising:
(a) obtaining a sample of antigen-containing blood and/or tissue;
(b) processing the blood and/or tissue to obtain a liquid or homogenate;
(c) adding to the liquid or homogenate sample at least one cross-linking agent;
(d) heating the sample to a temperature of over 50° C. until the sample solidifies;
(e) mixing the solidified sample with a pyrogen-free physiological solution to form a liquid antiviral agent.

2. The process of claim 1, wherein the sample comprises blood and step (b) is accomplished by agitation and/or by the addition of at least one coagulation inhibitor.

3. The process of claim 2, wherein the agitation is accomplished by shaking in the presence of glass pearls and by the addition of a pyrogen-free common salt solution.

4. The process of claim 1, wherein the at least one cross-linking agent of step (c) is selected from the group consisting of formaldehyde, p-formaldehyde, formalin, phenol, and phenol derivatives.

5. The process of claim 4, wherein the cross-linking agent is formalin.

6. The process of claim 5, wherein a saturated formalin solution is added in an amount of about 0.3 to 1.0 volume percent.

7. The process of claim 6, wherein a saturated formalin solution is added in an amount of about 0.5 volume percent.

8. The process of claim 1, wherein the pyrogen-free physiological solution of step (e) is a pyrogen-free physiological common salt solution.

9. The process of claim 1, wherein step (d) comprises heating the sample to a temperature of about 55 to about 85° C.

10. The process of claim 9, wherein the elevated temperature is maintained for about 2 hours.

11. The process of claim 1, further comprises filtering the liquefied antiviral agent through a narrow-pored filter.

12. The process of claim 11, wherein the filter is a sterile filter.

13. The process of claim 1, wherein the sample comprises blood and step (b) comprises:
(a) subjecting the blood sample to an erythrocyte lysis,
(b) centrifuging off the lymphocyte fraction, and
(c) resuspending the lymphocyte fraction in a physiological common salt solution or phosphate buffered saline.

14. The process of claim 1, wherein the antiviral agent is effective against a virus selected from the group consisting of HIV, papilloma, herpes, hepatitis C, and hepatitis B.

15. The process of claim 1, wherein the antiviral agent is used to treat a disease caused by a viral infection and selected from the group consisting of AIDS, Crohn's disease, tumors, and carcinomas.

16. The process of claim 1, wherein the sample comprises tissue and step (b) comprises:
(a) adding to the tissue sample a pyrogen-free physiologically acceptable aqueous dilution agent; and
(b) processing the tissue to obtain a homogenate.

17. The process of claim 16, wherein step (b) comprises:
(a) mechanically comminuting the tissue; and
(b) homogenizing the tissue.

18. The process of claim 16, wherein the pyrogen-free physiologically acceptable aqueous dilution agent is a pyrogen-free physiological common salt solution.

19. The process of claim 17, further comprising adding at least one coagulation inhibitor prior to homogenization.

20. The process of claim 17, wherein the mechanical comminution is accomplished by an ultrasonic comminuter.

21. Denatured antigens and viruses obtained according to claim 1.

22. An antiviral agent composition prepared according to the method of claim 1.

23. A method of treating a mammal in need with an antiviral composition according to claim 1 comprising administering an effective amount of the antiviral composition, wherein administration results in a decrease in the viral load of the mammal.

24. The method of claim 23, wherein the administration comprises multiple doses over a period of time.

25. The method of claim 23, wherein the mammal is a human.
